(19) United States
(12) Patent Application Publication
(10) Pub. No.: US 2011/0288124 A1
(43) Pub. Date: Nov. 24, 2011

(54) COMPOSITIONS AND METHOD FOR TREATMENT OF ATTENTION DEFICIT DISORDER AND ATTENTION DEFICIT/HYPERACTIVITY DISORDER WITH METHYLPHENIDATE

(75) Inventors: Juan Mantelle, Miami, FL (US); Terese A. Dixon, Miami, FL (US)

(73) Assignee: NOVEN PHARMACEUTICALS, INC.

(21) Appl. No.: 13/071,048

(22) Filed: Mar. 24, 2011

Related U.S. Application Data
(63) Continuation of application No. 12/981,154, filed on Dec. 29, 2010.
Application No. 12/981,154 is a continuation of application No. 11/812,198, filed on Jun. 15, 2007, now abandoned.
Application No. 11/812,198 is a continuation of application No. 11/378,629, filed on Mar. 20, 2006, now abandoned.
Application No. 11/378,629 is a continuation of application No. 10/024,513, filed on Dec. 21, 2001, now abandoned.
Application No. 10/024,513 is a continuation-in-part of application No. 09/618,626, filed on Jul. 18, 2000, now Pat. No. 6,348,211.
Application No. 09/618,626 is a division of application No. 09/163,351, filed on Sep. 30, 1998, now Pat. No. 6,210,705.
(60) Provisional application No. 60/069,510, filed on Dec. 15, 1997.

(51) Int. Cl. A61K 31/4458; A61P 25/00

(52) U.S. Cl. 514/317

(57) ABSTRACT

The invention relates to a method of treating Attention Deficit Disorder (ADD) and Attention Deficit/Hyperactivity Disorder (ADHD) and compositions for topical application of methylphenidate comprising methylphenidate in a flexible, finite system wherein said composition comprises about 10 to 30 wt % methylphenidate, about 30 to 50 wt % acrylic adhesive, and about 30 to 50 wt % silicone adhesive and wherein said methylphenidate is delivered to a subject in need thereof such that the plasma concentration of methylphenidate increases over a period of about 6-16 hours, and more preferably over a period of about 6-12 hours followed by a steady decrease in plasma concentration of methylphenidate.

This application is a continuation in part of U.S. patent application Ser. No. 09/618,626, filed Jul. 18, 2000, which is a Divisional application of U.S. patent application Ser. No. 09/163,351, filed Sep. 30, 1998, now U.S. Pat. No. 6,210,705, which claims the benefit of U.S. provisional Application Ser. No. 60/069,510, filed Dec. 15, 1997, now abandoned. These applications are hereby incorporated by reference in their entirety.

### BACKGROUND OF THE INVENTION

1. Field of the Invention

The present invention relates to compositions and methods for the treatment of Attention Deficit Disorder (ADD) and Attention Deficit/Hyperactivity Disorder (ADHD) by means of topical application of methylphenidate.

2. Background of the Invention

Attention Deficit Disorder (ADD) and Attention Deficit/Hyperactivity Disorder (ADHD) (severally and collectively hereinafter referred to as “AD”) are developmental disorders of self-control. They consist of problems with attention span, impulse control, and activity level. These problems are reflected in impairment of a person's will or capacity to control his or her own behavior relative to the passage of time and to keep future goals and consequences in mind.

Traditionally, methylphenidate has been used as the drug of choice for the treatment of AD in both children and adults for several reasons. Methylphenidate, described in U.S. Pat. No. 2,957,880, is a central nervous system stimulant. Though not an amphetamine, methylphenidate functions in a similar way in the brain. The current commercially available dosage form (Ritalin® tablets) and available strengths of the tablets fall short of providing effective treatment for a significant portion of the patient's waking hours. Methylphenidate has a short duration of action of from about 2 to 4 hours. A controlled release tablet of methylphenidate is commercially available, but is available only in one strength. This product, which was designed to eliminate the need for multiple administrations of a tablet during the school day for children and reduce dosing to either once or twice a day, falls short of providing effective treatment for a significant portion of the patient's waking hours.

Indeed, the regimen of methylphenidate currently used for ADHD exhibits numerous shortcomings that include fluctuations in blood levels with immediate release tablets; inconvenience of successfully complying with more frequent dosing (for examples, inability of children to accurately monitor time and/or stigma of medication); difficulty for young children to swallow tablets whole; availability of only two types of tablets available, immediate release tablets and sustained release tablets; ineffectiveness of BID (behavioral inhibition disorder) dosing for a significant portion of the patient's waking hours; and potential for drug abuse.

In addition, when methylphenidate is administered in a dosage form (immediate release tablets or sustained release tablets) it does not take into account the need for a “sleep window” in patients early on in treatment. There is a time frame referred to as a “sleep window,” which begins about 30 minutes prior to the end of the efficacy period for the preceding dose and extends from about 30 to 60 minutes beyond the end of the efficacy period for that dose. This gives a 60 to 90 minute period of time when the patient can lie down and drift into restful sleep. If the delay is longer, the rebound symptoms may be fully present, which then prevents a person from going to sleep. The result is an apparent over stimulation insomnia that is not related to too much medication, but to a drop in blood level of the medication. Rebounding is a return of the AD symptoms after the medication wears off. During this period of rebounding, the symptoms of AD may actually be worse than they were before dosing.

Topical application of drugs provides many advantages over conventional oral administration. Advantages include convenience, uninterrupted therapy, improved patient compliance, ease of discontinuance, elimination of hepatic first pass metabolism, a high degree of control over blood concentration of the drug and improved overall therapy.

The term “topical” or “topically” is used herein in its conventional meaning as referring to direct contact with a spot on a mammal, which can be any anatomical site or surface area including skin or mucous membranes, or hardened tissue such as teeth or nails.

The term “application” is intended to mean any mode that results in systemic administration.

The term “mucosa” or “mucosal” as used herein means oral, buccal, vaginal, rectal, nasal, intestinal, and ophthalmic surfaces.

Although topical application systems have many advantages, most drugs do not readily lend themselves to this mode of administration due to the well known barrier properties of the skin. Molecules moving from the environment into and through intact skin must first penetrate the stratum corneum, the outer horny layer of the skin, and any material on its surface. The molecule must then penetrate the viable epidermis and the papillary dermis before passing through the capillary walls and into the systemic circulation. Along the way, each of the above-mentioned tissues will exhibit a different resistance to penetration by the same molecule. However, it is the stratum corneum, a complex structure of compact keratinized cell remnants separated by extracellular lipid domains, that presents the greatest barrier to absorption of topical compositions or transdermally administered drugs.

There are topical application systems known in the art which provide a means for transdermal delivery of various drugs where methylphenidate is mentioned, e.g., in Quan et al., U.S. Pat. No. 5,601,839, a transdermal delivery system is disclosed. A basic drug having a pKa of 8.0 or greater is incorporated into the delivery system. The formulation also requires the use of triacetin as a permeation enhancer. Quan et al. lists oxybutynin, scopolamine, fluoxetine, epinephrine, morphine, hydromorphone, atropine, cocaine, buprenorphine, chlorpromazine, imipramine, desipramine, methylphenidate, methamphetamine, lidocaine, procaine, pindolol, nadolol, and carisoprodol as preferred “basic drugs.” Bloom et al., U.S. Pat. No. 5,614,178, discloses a composition for topical delivery comprising an effective amount of a pharmaceutically active substance, a high molecular weight crosslinked cationic polymer, a non-ionic surfactant, an alkoxylated ether, and a pharmaceutically acceptable carrier. Bloom et al. includes a myriad of different drugs for incorporation into the topical delivery system. Lee et al., U.S. Pat. No. 5,629,019 discloses a transdermal delivery composition containing a hydrophobic permeation enhancer, which permeation enhancer has been micronized and stabilized in an inert carrier. These compositions can include a biologically active substance to provide enhanced permeability of the active agent to the skin or mucosa. Lee et al. lists over 100 beneficial agents to be included in the transdermal delivery composition.

Nevertheless, one of the problems still associated with the administration of methylphenidate is the loss of efficacy when constant blood levels are maintained. Thus, methylphenidate formulations in which steady state values are rapidly achieved, for example in an hour or less, are less effective than those in which the plasma concentration increases over several hours to a steady state level, or even more effectively gradually decreases after peaking.

Therefore, despite the existence of many different types of topical application systems in the art, there remains a continuing need for improving the method of delivery of methylphenidate to a patient.

### SUMMARY OF THE INVENTION

It is therefore an object of the present invention to provide a composition for topical application of methylphenidate that overcomes the known disadvantages described above by delivering methylphenidate in an amount and at a rate sufficient to increase the plasma concentration of methylphenidate over a period of about 6-16 hours, followed by a steady decrease in the plasma concentration of methylphenidate.

To accomplish the foregoing and other objects of the invention, there has been provided according to one aspect of the invention, a composition for topical application of methylphenidate, that includes methylphenidate and a pharmaceutically acceptable adhesive in a flexible, finite system. The composition delivers methylphenidate in an amount and rate sufficient to increase the methylphenidate plasma concentration of a subject being treated over a period of about 6-16 hours, followed by a steady decrease in the plasma concentration of methylphenidate. In a preferred embodiment, the composition includes about 10 to 30 wt % methylphenidate, about 30 to 50 wt % acrylic adhesive, and about 30 to 50 wt % silicone adhesive.

According to another aspect of the invention, there has been provided, a method of treating attention deficit disorder and attention deficit/hyperactivity disorder that includes topically administering a composition of methylphenidate and a pharmaceutically acceptable adhesive in a flexible, finite system. The composition delivers methylphenidate in an amount and rate sufficient to increase the methylphenidate plasma concentration of a subject being treated over a period of about 6-16 hours, followed by a steady decrease in the plasma concentration of methylphenidate. In a preferred embodiment, the composition being topically applied includes about 10 to 30 wt % methylphenidate, about 30 to 50 wt % acrylic adhesive, and about 30 to 50 wt % silicone adhesive.

Further objects, features and advantages of the present invention will become apparent from detailed consideration of the preferred embodiments that follow.

### BRIEF DESCRIPTION OF THE DRAWINGS

FIG. 1 shows the linear plot of the mean d-methylphenidate plasma profiles in 29 subjects on day 6 after administering (a) methylphenidate in a 25 cm² transdermal composition having 20 wt % methylphenidate based on the entire weight of the composition every day over a period of 16 hours or (b) 20 mg of oral Ritalin® at 7 AM, 11 AM, and 3 PM daily. The graph demonstrates a continuous increase in the mean plasma concentration of methylphenidate over a period of about 6-12 hours followed by a steady decrease in the plasma concentration of methylphenidate.

FIG. 2 shows the linear plot of the mean linear /-methylphenidate plasma profiles in 29 adult subjects on day 6 after administering (a) methylphenidate in a 25 cm² transdermal composition having 20 wt % methylphenidate based on the entire weight of the composition every day over a period of 16 hours or (b) 20 mg of oral Ritalin® at 7 AM, 11 AM, and 3 PM daily. The graph demonstrates a continuous increase in the mean plasma concentration of methylphenidate over a period of at least 8 hours.

### DETAILED DESCRIPTION OF THE PREFERRED EMBODIMENTS

Methylphenidate has the following general formula:

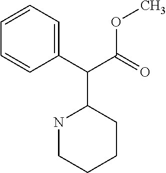

There are four enantiomers which are the (2R:2′R)-(+)-threo-enantiomer, the (2S:2′S)-(−)-threo-enantiomer, the (2R:2′S)-(+)-erythro-enantiomer, and the (2S:2′R)-(−)-erythro-enantiomer, but only the d-threo-methylphenidate is significantly active. Commercially available Ritalin is 50:50 d-threo-methylphenidate:/-threo-methylphenidate. The degradation products or metabolites of methylphenidate are also essentially inactive.

Equivalent to the base methylphenidate for the purpose of this invention are the pharmaceutically acceptable acid addition and quaternary salts of the base methylphenidate. Particularly suitable are salts of weak acids. A variety of inorganic and organic acids form pharmaceutically acceptable salts of methylphenidate. The salts are formed with acids such as sulfuric, phosphoric, hydrochloric, hydrobromic, hydriodic, sulfamic, citric, lactic, maleic, malic, succinic, tartaric, cinnamic, acetic, benzoic, gluconic, ascorbic, and related acids. It is also possible to form quaternary ammonium salts with a variety of organic esters of sulfuric, hydrohalic, and aromatic sulfonic acids. Among such esters are methyl chloride and bromide, ethyl chloride, propyl chloride, butyl chloride, isobutyl chloride, benzylchloride and bromide, phenethyl bromide, naphthymethyl chloride, dimethyl sulfate, methyl benzenesulfonate, ethyl toluenesulfonate, ethylene chlorohydrin, propylene chlorobydrin, allyl bromide, methylallyl bromide and crotyl bromide.

The amount of methylphenidate can range from 5 to 35 wt %, more preferably 10 to 30 wt %, and still more preferably 15 to 35 wt % based on the entire weight of the composition.

Particularly preferred carriers are pressure-sensitive adhesive flexible, finite carriers. These can include any viscoelastic material which adheres instantaneously to most substrates with the application of very slight pressure and remains permanently tacky. A polymer is a pressure-sensitive adhesive within the meaning of the term as used herein if it has the properties of a pressure-sensitive adhesive per se or functions as a pressure-sensitive adhesive by admixture with tackifiers, plasticizers or other additives. The term pressure-sensitive adhesive also includes mixtures of different polymers and mixtures of polymers, such as polyisobutylenes (PIB), of different molecular weights, wherein each resultant mixture is a pressure-sensitive. Other useful rubber based pressure-sensitive adhesives include hydrocarbon polymers such as natural and synthetic polyisoprene, polybutylene and polyisobutylene, styrene/butadiene polymers styrene-isoprene-styrene block copolymers, hydrocarbon polymers such as butyl rubber, halogen-containing polymers such as polyacrylic-nitrile, polytetrafluoroethylene, polyvinylchloride, polyvinylidene chloride, and polychlorodiene, and other copolymers thereof. Particularly suitable bioadhesives or mucoadhesives include natural or synthetic polysaccharides and polyacrylic acid polymers, and mixtures thereof. The term “polysaccharide” as used herein means a carbohydrate decomposable by hydrolysis into two or more molecules of monosaccharide or their derivatives. Preferred polysaccharides include cellulose materials and natural gums. Such adhesives may be used singularly, or in blends of two or more, or in combination (i.e., in layers).

Other useful pressure-sensitive adhesives (“PSA”) can include acrylic-based pressure-sensitive adhesives and silicone-based pressure-sensitive adhesives as described in U.S. Pat. Nos. 5,474,783, and 5,656,386. Suitable commercially available acrylic-based polymers can include adhesives that are commercially available and include the polyacrylate adhesives sold under the trademarks Duro-Tak by National Starch and Chemical Corporation, Bridgewater, N.J., such as Duro-Tak 87-2194, Duro-Tak 87-2196, Duro-Tak 87-1197, 87-4194, 87-2510, 87-2097 and 87-2852. Other suitable acrylic-based adhesives are those sold under the trademarks Gelva-Multipolymer Solution (GMS) (Monsanto; St. Louis, Mo.), such as GMS 737, 788, 1151, 3087 and 7882.

Suitable silicone-based pressure-sensitive adhesives can include those described in Sobieski, et al., “Silicone Pressure Sensitive Adhesives,” Handbook of Pressure-Sensitive Adhesive Technology, 2nd ed., pp. 508-517 (D. Satas, ed.), Van Nostrand Reinhold, New York (1989), incorporated by reference in its entirety. Other useful silicone-based pressure sensitive adhesives are described in the following U.S. patents: U.S. Pat. Nos. 4,591,622; 4,584,355; 4,585,836; and 4,655,767. Suitable silicone-based pressure-sensitive adhesives are commercially available and include the silicone adhesives sold under the trademarks BIO-PSA 7-4503, BIO-PSA 7-4603, BIO-PSA 7-4301, 7-4202, 7-4102, 7-4106, and BIO-PSA 7-4303 by Dow Corning Corporation, Medical Products, Midland, Mich.

The amount of the polymer carrier can range from 2 to 99 wt %, preferably, 30 to 90 wt %, even more preferably 50 to 90 wt %, still more preferably 75 to 85 wt % based on the entire weight of the composition.

In a particularly preferred embodiment of the invention, the multiple polymer adhesive system comprises a pressure-sensitive adhesive blend of an acrylic-based polymer, a silicone-based polymer, and optionally a soluble PVP (described below). For these embodiments to achieve the desired flux, the acrylic-based polymer and silicone-based polymer are generally present in an amount of from 20 to 80 wt % and 10 to 50 wt %, more preferably in an amount of from 30 to 50 wt % and 20 to 45 wt % respectively, based on the entire weight of the composition, and still more preferably 35 to 45 wt % and 30 to 40 wt %. The amount of acrylic-based (also referred to broadly as a polyacrylate) polymer and silicone-based polymer (also referred to broadly as a polysiloxane) may be adjusted so as to modify the saturation concentration of methylphenidate in the multiple polymer adhesive system in order to affect the rate of delivery of methylphenidate from the system and through the skin while still maintaining the desired plasma profile. Other useful ranges include about 5-85% by weight of the acrylate-based polymer, 10-90% by weight of polyisobutylene and 5-95% by weight of silicone-based polymer.

It has been discovered that methylphenidate, and in particular the base form, can be unstable and undergoes degradation in the presence of acid functional groups which are contained in adhesives, enhancers, excipients and other components of the topical composition. The major degradant/metabolite appears to be ritalinic acid, which increases about ten fold with every 1% increase by weight in such acid functional component. Such degradation can greatly reduce the amount of the active enantiomer during storage of the topical composition, thus reducing the amount of active methylphenidate available for drug delivery.

In view of the foregoing, polymers, particularly acrylic polymers that are non-functional, hydroxy functional, or minimally acid functional are preferred. A “minimally acid functional polymer” (e.g. acrylic) is defined as a polymer (e.g. acrylic) having no more than about 5 wt % of acid functional monomers, preferably no more than about 1 wt %, and more preferably no more than about 0.6 wt % of acid functional monomer, based on the weight of the polymer (e.g. acrylic). Likewise, other components of the composition contain less than 5 wt %, preferably less than about 1 wt %, more preferably less than about 0.6 wt % acid functional groups based on the weight of the composition.

Further instability, in terms of a yellowing color change which may be undesirable in a finished product, has been observed in the presence of vinyl acetate. Thus, while vinyl acetate and adhesives containing vinyl acetate monomer units, such as ethylene/vinyl acetate copolymers, and vinyl pyrrolidone/vinyl acetate, have been found to work satisfactorily, the use of these is generally not as preferred as the other adhesives listed above.

It has further been discovered that use of capped (or amine-compatible) polysiloxanes also increase stability and reduce degradation in topical compositions. In addition to reducing the amount of the ritalinic acid, it appears that such polysiloxane polymers reduce the overall reactivity of the composition and therefore the appearance of other degradation products such as the erythro-enantiomers. A “capped” polysiloxane polymer is one which has been chemically treated to reduce or eliminate the silicone-bonded hydroxyl content preferably by substitution with a hydrocarbon radical such as a methyl group. Illustrative examples of capped polysiloxanes include those described in U.S. Pat. No. Re. 35,474, incorporated herein by reference, and which are commercially available from Dow Corning Corporation under their BIO-PSA 7-4100, -4200 and -4300 product series.

The phrase “flexible, finite system” is intended to mean a solid form capable of conforming to the surface with which it comes into contact, and which is capable of maintaining the contact in such solid form so as to facilitate topical application without adverse physiological response, and without being appreciably decomposed by aqueous contact during administration to a patient.

Illustrative examples of suitable adhesives and flexible, finite delivery systems include those described in U.S. Pat. Nos. 5,474,783, and 5,656,386 both assigned to Noven Pharmaceuticals, Inc., Miami, Fla. (incorporated herein by reference).

Other flexible, finite systems known in the art include films, plasters, dressings, and bandages, as well as multilayer delivery systems in which the drug is solubilized or contained in one or more separate layers and reservoir-type delivery systems in which the drug is solubilized or contained in a reservoir or depot separate form the adhesive which attaches directly to the skin or mucosa.

In addition, the solubility of the methylphenidate can be altered by the optional addition of an agent that increases the solubility of methylphenidate in the topical application system, such as polyvinylpyrrolidone.

Of course the composition according to the present invention can also contain agents known to accelerate the delivery of a drug through the skin. These agents have been referred to as skin-penetration enhancers, accelerants, adjuvants, and sorption promoters, and are herein referred to collectively as “enhancers.” This class of agents includes those with diverse mechanisms of action including those which have the function of improving the solubility and diffusibility of a drug within the multiple polymer and those which improve percutaneous adsorption, for example, by changing the ability of the stratum corneum to retain moisture, softening the skin, improving the skin's permeability, acting as penetration assistants or hair-follicle openers or changing the state of the skin including the boundary layer. Some of these agents have more than one mechanism of action, but in essence they serve to enhance the delivery of a drug.

Some examples of enhancers are polyhydric alcohols such as dipropylene glycol, propylene glycol, and polyethylene glycol which enhance drug solubility; oils such as olive oil, squalene, and lanolin; fatty ethers such as cetyl ether and oleyl ether; fatty acid esters such as isopropyl myristate which enhance drug diffusibility; urea and urea derivatives such as allantoin which affect the ability of keratin to retain moisture; polar solvents such as dimethyldecylphosphoxide, methyloctylaulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

According to the present invention, methylphenidate may be administered to the human body via topical application delivery to the skin or mucosa for the purpose of treating AD in a composition that results in a continuously increasing methylphenidate plasma concentration over a period of about 6-16 hours and preferably about 6-12 hours, followed by a steady decrease in the plasma concentration of methylphenidate, preferably decreasing over a period of at least 8 hours. Other suitable period of increasing methylphenidate concentration include 8-16 hours.

The present composition provides a release of methylphenidate to the patient via topical application route. A delivery rate of about 0.5 mg/24 hours to about 100 mg/24 hours of methylphenidate, and more preferably from about 7.5 mg/24 hours to about 60 mg/24 hours, is needed to achieve a therapeutically effective dose in a patient. The topical application system may contain between about 15 mg to 110 mg of methylphenidate or an effective amount which will not crystallize in the system. The size of the delivery patch is in the range of from about 2 cm² to about 60 cm². The preferred system of this invention delivers about 0.5 mg per 24 hours and contains about 2.0 mg of methylphenidate base per cm².

As used herein, the term, “flux” is defined as the absorption of the drug through the skin or mucosa, and is described by Fick's first law of diffusion:

J=−D(dCm/dx),

where J is the flux in g/cm²/sec, D is the diffusion coefficient of the drug through the skin or mucosa in cm²/sec and dcm/dx is the concentration gradient of the drug across the skin or mucosa.

The inventors have found that there is a relatively wide range of permeability of normal human skin to methylphenidate and this permeability not only varies from individual to individual and site to site, but also is dependent upon the chemical form of the drug. It is preferred that the methylphenidate in the topical application system be in the base form or a base/basic salt combination, or an ester.

As used herein, the term “therapeutically effective dose” intends that dose of methylphenidate that achieves a therapeutic effect, and is typically in the range of about 0.05 mg/kg to about 1.0 mg/kg/day for both children and adults, and more preferably of about 0.075 mg/kg/day to about 0.3 mg/kg/day.

Attainment of continuously increasing methylphenidate plasma concentration over a period of about 6-16 hours and followed by a steady decrease in the plasma concentration of methylphenidate is ensured by providing enough methylphenidate in the topical composition so as to deliver 15% to 40% of the drug in the first 10 hours. A preferred embodiment for attaining continuously increasing methylphenidate plasma concentration over a period of about 6-12 hours followed by a steady decrease in the plasma concentration of methylphenidate is to include in the composition the polymers described above, such as the acrylics having no or minimal functional groups, or the capped silicone polymers. Use of such polymers assists in allowing sufficient amounts of methylphenidate to be loaded into the composition, while preserving the methylphenidate in the active form needed for continuously increasing methylphenidate plasma concentration over a period of about 6-12 hours and followed by a steady decrease in the plasma concentration of methylphenidate.

The invention contemplates the delivery of methylphenidate in therapeutic amounts for continuous periods in topical application systems that rely primarily on skin or mucosa permeability to control drug input rate. It is also contemplated that delivery of the drug can be from a rate controlled system in which the system itself controls the maximum rate at which the drug is delivered through the skin or mucosa.

The rate of increase in methylphenidate plasma concentration varies broadly and will depend, in part, on the size of the patch being applied. That is, for smaller patches such as 6.25, 12.5 or 18.75 cm² patches, the rate of increase will typically be lower, whereas for larger patches such as 25, 37 or 50 cm² patches, the rate of increase will typically be higher. The rate of increase can vary from a minimum of about 0.06 ng/ml/hr (with a 6.25 cm² patch) to a maximum of about 6 ng/ml/hr (with a 50 cm² patch). For a 25 cm² patch, the rate of increase in the methylphenidate plasma concentration is preferably in the range of 0.4 ng/mL/hr to 2.5 ng/mL/hr.

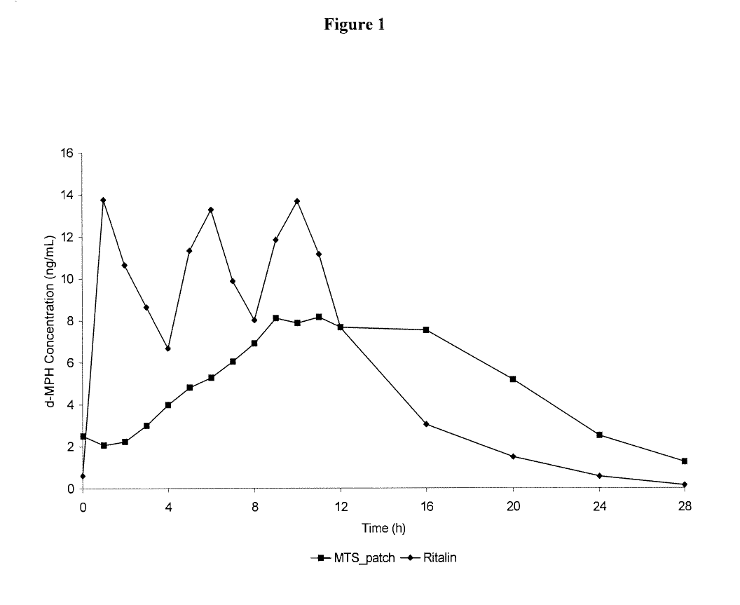

As used herein “steady decrease” encompasses the plasma profile of methylphenidate after the increase over the period of 6-16 hours. The steady decrease in the plasma profile may be constant for short periods of time, such as shown in FIG. 1, or even slightly increase. All that is required is that, on average, there is a decrease in the plasma levels of methylphenidate after the increase of the period of about 6-16 hours. Preferably, the steady decrease will be for 6 hours and more preferably 8 hours.

In some instances, the steady decrease may be broadly considered, “substantially zero-order” as that term is used in co-owned Ser. No. 09/161,351, from which this application claims priority, in that the variability contemplated within the scope of “substantially zero order” of about a 30% to about 40% difference from the mean in the plasma levels of methylphenidate at steady state (6-16 hours after administration) would also include a 30 to 40% decrease from the mean plasma levels of methylphenidate.

### Example 1

The following example are included as illustrative of topical application systems and compositions within the contemplation of the invention. This example are in no way intended to be limiting of the scope of the invention.

The topical delivery composition was prepared as follows: A mixture of 33 parts of a polysiloxane adhesive (BIO-PSA 7-4102), 53 parts of a polyacrylate adhesive (Gelva 3087), 3 parts of ethyl acetate and 10 parts of methylphenidate are added, the mixture in a vessel is agitated until a homogenous mixture is formed. The mixture is then coated on a release liner, the unit is then passed through an oven in order to drive off the volatile solvents. The resulting composition on a dry w/w % is 40 parts polysiloxane adhesive, 40 parts polyacrylate adhesive, and 20 parts methylphenidate. Upon completion of this step, the adhesive-drug component layer is joined to a backing material and the unit is wound into rolls for storage.

Methylphenidate flux through cadaver skin in vitro from the above formulation shows a skin permeability of 5 μg/cm²/hr to 40 μg/cm²/hr.

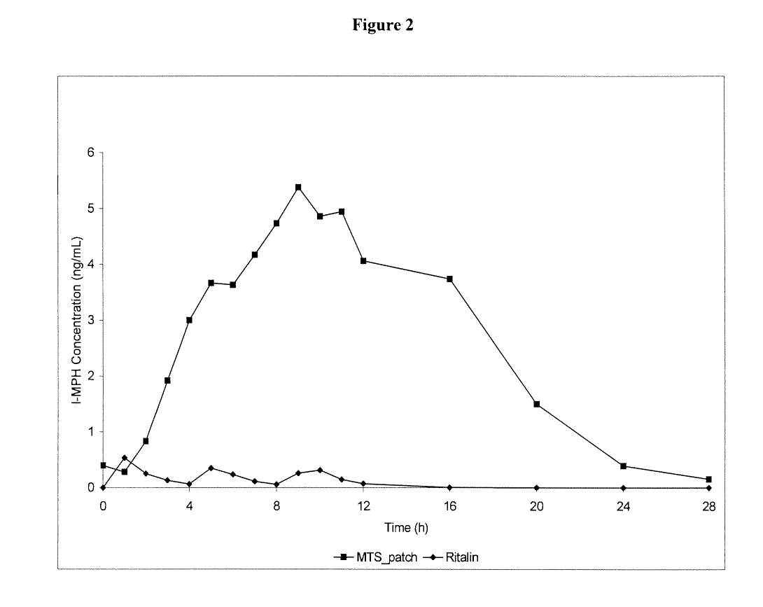

To study the pharmacokinetics of methylphenidate after dosing with the transdermal composition of Example 1 and Ritalin®, twenty-nine normal, healthy, non-smoking male and female subjects between the ages of 21-41 were randomized to receive either (a) a 25 cm² transdermal composition according to Example 1 for 16 hours a day, beginning at 7 AM, for 6 days, or (b) Ritalin® 20 mg tablets, 3 times a day (7 AM, 11 AM, 3 PM) for 6 days. Each subject then had a non-medication period of 7 days (“washout” period) followed by receiving the other dosage form. During each treatment period, blood samples (5 ml) were collected into chilled evacuated glass tubes containing EDTA (ethylene diamine tetraacetic acid) at point 0 (pre-dose) on days 4, 5, and 6. On day 6, additional samples were collected at points 1, 2, 3, 4, 5, 6, 7, 8, 9, 10, 11, 12, 16, 20, 24 and 28 hours post-dose. Plasma harvested from these blood samples were used to assay for d-threo-methylphenidate and I-threo-methylphenidate plasma concentrations, which are depicted in FIGS. 1 and 2.

Additional advantages, features and modifications will readily occur to those skilled in the art. Therefore, the invention in its broader aspects is not limited to the specific details, and representative devices, shown and described herein. Accordingly, various modifications may be made without departing from the spirit or scope of the general inventive concept as defined by the appended claims and their equivalents.

As used herein and in the following claims, articles such as “the,” “a” and “an” can connote the singular or plural.

All documents referred to herein are specifically incorporated herein by reference in their entireties.

1. A transdermal methylphenidate composition, comprising methylphenidate in a pharmaceutically acceptable pressure-sensitive adhesive carrier, wherein said composition transdermally delivers methylphenidate in an amount and at a rate sufficient to achieve a methylphenidate plasma concentration that increases over a period of at least about 6 hours, followed by a steady decrease.

2. The composition according to claim 1, wherein said steady decrease occurs over a period of at least about 8 hours.

3. The composition according to claim 1, wherein said methylphenidate plasma concentration increases over a period of about 6 to about 12 hours.

4. The composition according to claim 1, wherein said methylphenidate plasma concentration increases at a rate of from 0.06 (ng/mL)/hour to 6.0 (ng/mL)/hour.

5. The composition according to claim 1, wherein said methylphenidate plasma concentration increases at a rate of from 0.4 (ng/mL)/hour to 2.5 (ng/mL)/hour.

6. The composition according to 1, wherein said composition comprises no more than about 5% weight/weight of acid functional monomers.

7. The composition according to claim 1, wherein said composition is substantially free of ritalinic acid at the time of manufacture.

8. The composition according to claim 1, wherein said composition delivers a therapeutically effective amount of methylphenidate over a period of time of about 12 to about 24 hours.

9. The composition according to claim 1, wherein said composition delivers a therapeutically effective amount of methylphenidate over a period of time of about 12 to about 18 hours.

10. The composition according to claim 1, wherein said composition delivers methylphenidate at a rate of at least 5 mg per 24 hours.

11. The composition according to claim 1, comprising about 10 to 30 wt % methylphenidate, about 30 to 50 wt % acrylic adhesive, and about 30 to 50 wt % silicone adhesive.

12. (canceled)

13. The composition according to claim 1, wherein said methylphenidate plasma concentration occurs over a period of about 6 to about 16 hours.

14-19. (canceled)

20. A method of treating attention deficit disorder and/or attention deficit/hyperactivity disorder comprising topically administering a composition according to claim 1.

21-38. (canceled)

39. The composition according to claim 1, wherein the pharmaceutically acceptable pressure-sensitive adhesive carrier comprises a non-functional acrylic polymer.

40. The composition according to claim 39, comprising 5 to 35 wt % methylphenidate, based on the total weight of the composition.

41. The composition according to claim 39, further comprising a capped or amine-compatible polysiloxane polymer.

42. The composition according to claim 40, consisting essentially of said methylphenidate, said non-functional acrylic polymer and said capped or amine-compatible polysiloxane polymer.

43. The composition according to claim 39, wherein the composition does not include an enhancer.

44. The composition according to claim 1, wherein the methylphenidate is methylphenidate base.

45. The composition according to claim 1, wherein the methylphenidate comprises the d-threo-methylphenidate enantiomer.

46. The composition according to claim 1, wherein the methylphenidate consists essentially of the d-threo-methylphenidate enantiomer.

47. The composition according to claim 1, wherein the composition does not include vinyl acetate or vinyl acetate monomers.

48. The composition according to claim 1, wherein the methylphenidate is present in an amount effective to deliver from 3.96 mg to 10.56 mg methylphenidate per 10 cm² of said flexible finite, system within the first 10 hours of delivery.

49. The composition according to claim 1, wherein the methylphenidate is present in an amount effective to deliver from 23.76 to 63.36 mg methylphenidate within the first 10 hours of delivery.

50. The composition according to claim 1, wherein the methylphenidate is present in an amount effective to deliver from 9 to 24 mg methylphenidate within the first 10 hours of delivery.

51. The composition according to claim 1, wherein the methylphenidate is present in an amount of at least 26.4 mg per 10 cm² of said flexible finite system.

52. The composition according to claim 1, wherein the methylphenidate is present in an amount of about 26.4 mg per 10 cm² of said flexible finite system.

53. The composition according to claim 1, wherein the methylphenidate is present in an amount of about 10 mg/cm² of said flexible finite system.

54. The composition according to claim 1, wherein the methylphenidate is present in an amount of about 3 mg/cm² of said flexible finite system.

55. The composition according to claim 1, wherein the methylphenidate is present in an amount of about 0.33 mg/cm² of said flexible finite system.
